(19) United States
(12) Patent Application Publication
(10) Pub. No.: US 2004/0091418 A1
(43) Pub. Date: May 13, 2004

(54) Production of hydrogen

(76) Inventors: John Sydney Carlow, Hampshire (GB); Stephen Ivor Hall, Oxfordshire (GB); Anthony Robert Martin, Oxfordshire (GB)

Correspondence Address: William H Holt, Law Offices of William H Holt, First Floor Unit 2, 1423 Powhatan Street, Alexandria, VA, 22314, US

(21) Appl. No.: 10/472,206

(22) PCT Filed: Mar. 15, 2002
(86) PCT No.: PCT/GB02/01224

(30) Foreign Application Priority Data
Mar. 21, 2001 (GB) 0107020.0
Oct. 25, 2001 (GB) 0123379.3

(51) Int. Cl. B01J 19/08; C07C 1/00

(52) U.S. Cl. 423/650; 422/186.04; 204/168

(57) ABSTRACT

Hydrogen is generated from hydrocarbon fuel by a reforming reaction in a reactor (11) in which gaseous hydrocarbon and reforming reaction agents are subjected to the formation of a plasma having an electric field strength within the plasma exceeding 10,000 volts per centimetre, preferably at least 20,000 volts/cm, more preferably at least 40,000 volts/cm or even 80,000 volts/cm. The apparatus has electrodes (14, 15, 16) and dielectric material (22-27), the configuration of which allows for plasma having the electric field strength defined above.

The invention relates to the production of hydrogen from hydrocarbon fuel in a process which makes use of a non-thermal plasma reactor.

Fuel cells consuming hydrogen and oxygen offer the promise of providing clean power for motor vehicles. However, this leads to a requirement for an efficient and correspondingly clean process for the production of hydrogen. Ideally this should come, at least initially, from hydrocarbons such as are made available in quantities and on an appropriately wide distribution network in the form of gasoline or diesel for internal combustion engines.

The main routes known for the production of hydrogen from hydrocarbons are steam reforming, partial oxidation and carbon dioxide reforming.

Steam reforming involves reaction with water in the form of steam, sometimes referred to as “water shift” and may be represented as

CₘHₙ+mH₂O [symbol] mCO+(n/2+m)H₂, or

CₘHₙ+2mH₂O [symbol] mCO₂+(n/2+2m)H₂,

where m and n are the numbers of carbon and hydrogen atoms in a hydrocarbon molecule. These reactions are endothermic and heat energy must be provided to the gaseous reactants to satisfy thermodynamic constraints.

A reverse water shift reaction can also take place

CO+H₂O [symbol] CO₂+H₂

and this reaction is quasi-athermic.

In partial oxidation, reaction conditions have to be set up for a hydrocarbon to react with oxygen according to the equations

CₘHₙ+(m/2)O₂ [symbol] mCO+(n/2)H₂, or

CₘHₙ+([3m+1]/2)O₂ [symbol] mCO₂+(m+1)H₂O,

or with air

CₘHₙ+(m/2)O₂+(4m/2)N₂ [symbol] mCO+(n/2)H₂+(4m/2)N₂

CₘHₙ+([3m+1]/2)O₂+(4m/2)N₂ [symbol] mCO₂+(m+1)H₂O+(4m/2)N₂.

The partial oxidation reactions are highly exothermic, and so it is possible to use them in modes where less energy is required compared to steam reforming, thus minimising the power requirements. It also, of course, eliminates the need for water.

Carbon dioxide reforming is used to produce process gas with varying H₂:CO ratios and involves reaction of hydrocarbons with CO₂ (rather than with water)

CₘHₙ+mCO₂ [symbol] 2mCO+(n/2)H₂.

This reaction is endothermic so that heat must be supplied.

Apparatus and processes for performing hydrocarbon reforming reactions with assistance from plasma, both thermal and non-thermal have been described in a number of publications, examples of which are patent specification WO 98/28223, SAE Paper No. 982427, October 1998; Int.J.Hydrogen Energy 23,1109, 1998, patent specifications U.S. Pat. No. 5,993,761, U.S. Pat. No. 6,007,742, WO 01/00310, WO 01/09031, WO 01/14702.

In all these prior art examples, however, the plasma used can only supply a fraction of the energy required to achieve an efficient level of reforming and it is necessary to provide heating to supply at least 80% and more typically 90% or more of the total energy input required. There are a number of disadvantages to these prior art examples. The reactors must be built to operate at elevated temperatures and the materials used in their construction must have suitable mechanical properties for these elevated temperatures. The reaction chambers must be insulated to minimise heat losses. Burners or heat exchangers must be incorporated, adding to the complexity of the devices. There will be a period of time after turn-on over which hydrogen yields and power conversion efficiencies gradually increase from initially low levels to their final levels as the reactor warms up. Change of reactor output will be slow as the thermal response of a reactor will be long. Thus there are requirements for improved apparatus for the plasma-forming of hydrocarbons.

It is an object of the present invention to reduce and, if possible, eliminate the need to supply additional heat energy.

According to the invention there is provided an apparatus for performing hydrocarbon reforming reactions, which apparatus comprises a reactor chamber having an inlet duct for feeding gaseous reagents to the chamber and an outlet duct for reaction products, and, within the chamber, electrodes and dielectric material, a source of electrical potential connected in use of the apparatus to apply an electrical potential across the electrodes, the spacing and configuration of the electrodes and the dielectric material in relation to the applied electrical potential in use being such as to generate plasma in gaseous reagents passing through the reactor, in which plasma the electric field strength is in excess of 10,000 volts per centimetre, preferably at least 20,000 volts/cm, more preferably at least 40,000 volts/cm, and may desirably be at least 80,000 volts/cm.

To achieve such high electric field strengths within the plasma, the reactor is preferably a dielectric barrier (or so called “silent discharge”) reactor having at least one electrode with a dielectric barrier layer in intimate contact with its surface or surfaces which face the other electrode, and the spacing between the electrodes is sufficiently small for the applied potential across them to generate plasma having the aforesaid electric field strength. The reactor may be provided with a plurality of electrodes arranged in pairs and electrical potential is applied across each pair. Conveniently, all such electrodes are coated with a dielectric barrier layer.

In an alternative arrangement a bed of high permittivity dielectric particles fills the space between electrically conducting surfaces of the electrodes, the permittivity of the particles serving to concentrate the electric potential at the contact points between the particles such that the aforesaid electric field strength is achieved in plasma discharges formed in the interstices between the particles.

In a further alternative the electrodes, or each pair of electrodes, are, or is, provided with a dielectric barrier layer between them and the space between the electrodes, or each pair of electrodes is additionally filled with a bed of high permittivity dielectric particles.

We have also found that enhanced formation of discharge streamers emanating from dielectric surfaces, is achieved by providing, on those dielectric surfaces exposed to gas passing through the reactor, an isolated discontinuous metallic film, preferably less than 10 μm in thickness and not connected to an external source of electric potential. The discontinuous metallic film may be in the form of spots or dots of metal and can be present on one or more of the dielectric barriers. The metal provides a richer source of electrons than the dielectric material and has the effect of increasing the number of discharge points emanating from one or more of the barriers and thereby increasing the efficiency of treatment of gaseous reagents passing through the reactor.

Alumina is a suitable material for the dielectric barrier layer although other dielectric ceramic materials can be used. Barium titanate, calcium titanate or high permittivity dielectric material including ferroelectric materials are suitable materials for the particles in a bed reactor. The particles in a bed reactor may be in the form of spheres, pellets, extrudates, fibres, sheets, wafers, frits, meshes, coils, foams, honeycomb monoliths or granules or as a coating on any of the above shapes or contained within a dielectric, polymeric or metallic material in any of the above shapes or as a combination of more than one of the aforementioned forms of particulate material.

Preferably the apparatus includes a membrane positioned to receive output gaseous product from the reactor chamber to separate hydrogen gas therefrom. The membrane may be outside the reactor chamber or may be positioned within the reactor chamber and may be exposed to the plasma, in which case the plasma may serve to clear any fouling from the membrane. The membrane may conveniently be provided by one or more of the electrodes in the reactor chamber, which electrode or electrodes is or are in the form of a gas permeable electrically conducting material which itself is permeable to hydrogen but not other reaction products, or in the form of a gas permeable electrically conducting material having a coating thereon of material which is permeable to hydrogen but not other reaction products. Depending upon its location, the membrane may comprise a ceramic such as zirconia or alumina, or may be polymeric or metallic. The membrane pore size is preferably less than 2 nanometres. Location of the membrane within an electric field, specifically such as the electric field in the plasma, may enhance the membrane performance. The membrane may be coated with materials, such as metals, for example, palladium or titanium, or metal oxides which serve to enhance the separation of hydrogen from other reaction products, or alternatively or additionally which serve to store hydrogen.

Pressure swing absorption apparatus can be used as an alternative to membrane processes for hydrogen separation as indicated in U.S. Pat. No. 5,932,181 (Yukong Limited). Here the gaseous stream leaving the non-thermal plasma reactor can contain hydrogen, carbon monoxide, carbon dioxide, water and unreacted hydrocarbon. Gaseous components other than hydrogen are absorbed on a bed of absorbent in the pressure swing absorption apparatus, such as molecular sieve. Alternative cycles of operation of the apparatus at atmospheric pressure and vacuum or reduced pressure can selectively absorb gaseous components other than hydrogen.

Catalytic materials may be incorporated in the apparatus to assist the desired reforming reactions. Such catalytic materials may be positioned within the plasma region, or so as to act upon gaseous medium leaving the reactor chamber. Depending upon the required location, the catalytic material may be provided separately as particles in a bed or a coating on a substrate through which the gaseous medium is passed, or in the form of a coating upon the electrodes, upon the dielectric barrier material, upon dielectric bed particles, or upon the membrane. Suitable catalytic materials are nickel, palladium, platinum, ruthenium or rhodium that can also be supported on an oxide support, preferably rhodium or platinum on alumina.

The invention includes a method of reforming gaseous hydrocarbon, which method comprises passing gaseous reactants comprising hydrocarbon together with steam or carbon dioxide and/or oxygen through a reactor chamber having an inlet duct for feeding gaseous reagents to the chamber and an outlet duct for reaction products, and, within the chamber, electrodes and dielectric material, applying an electrical potential across the electrodes, the applied electrical potential in relation to the spacing and configuration of the electrodes and the dielectric material being such as to generate plasma in the gaseous reactants passing through the reactor, in which plasma the electric field strength is in excess of 10,000 volts per centimetre, preferably at least 20,000 volts/cm, more preferably at least 40,000 volts/cm, and may desirably be at least 80,000 volts/cm.

Gaseous hydrocarbons that are to undergo reforming reactions can be derived from hydrocarbons that are in the gaseous, liquid or solid state at normal ambient temperatures.

Specific constructions of reactor for carrying out the invention will now be described by way of example and with reference to the drawings filed herewith, in which:

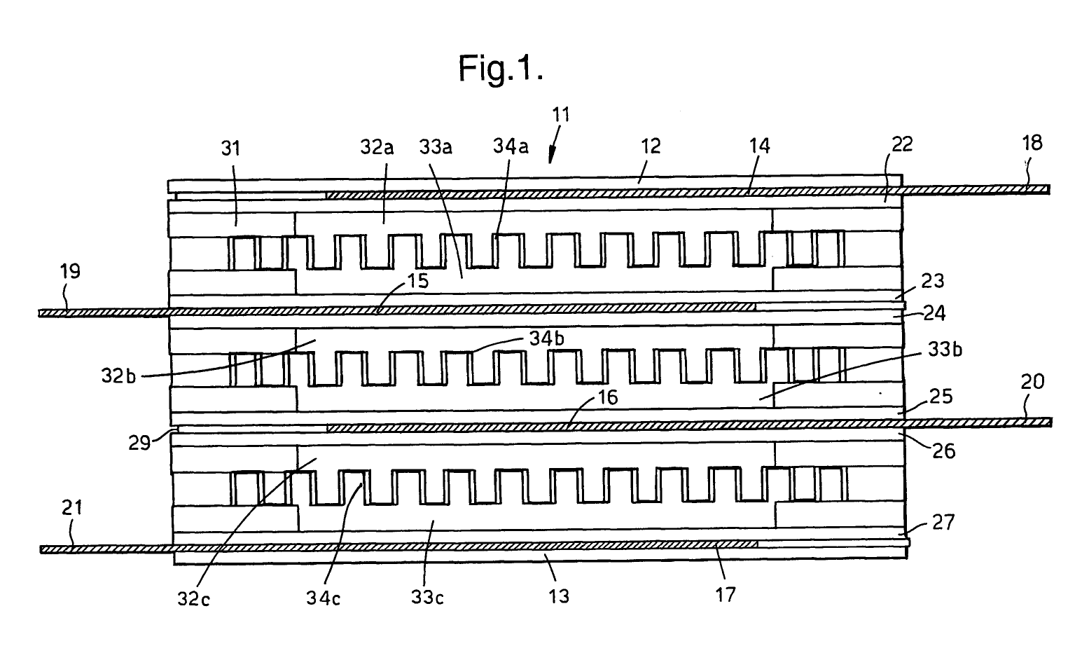

FIG. 1 shows a reactor in cross-section taken transverse to the direction of incoming and out-flowing gas,

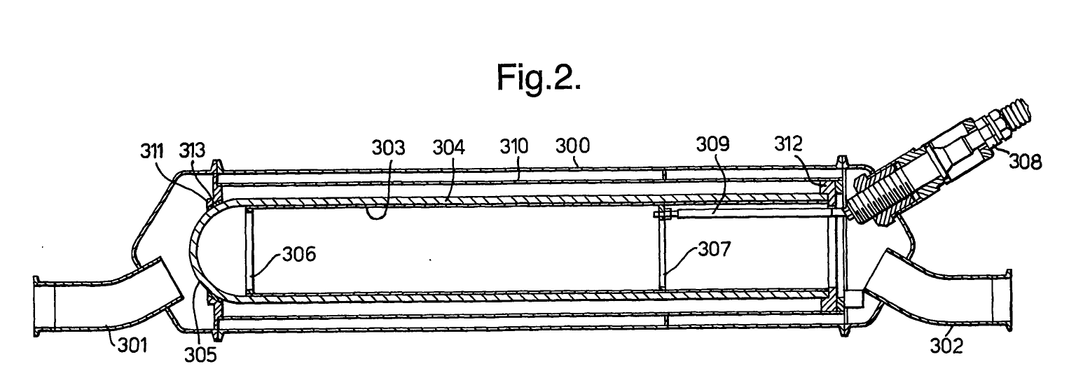

FIG. 2 is a longitudinal section of a second reactor, and

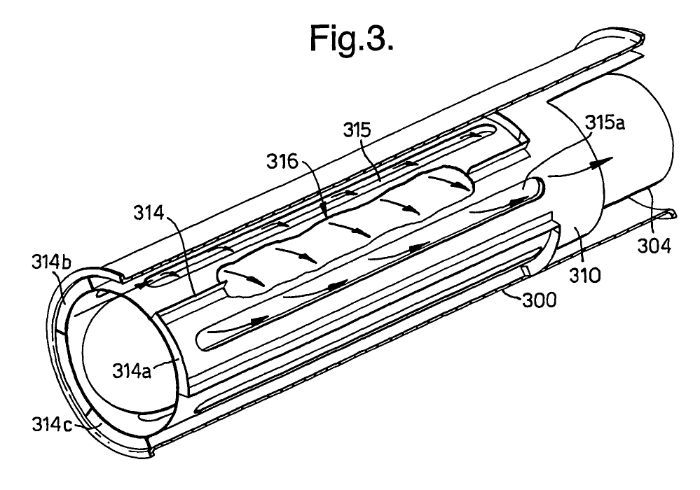

FIG. 3 is a schematic view showing the gas flow path through the reactor of FIG. 2.

A number of known forms of non-thermal plasma reactor are suitable for carrying out the present invention. As indicated above, these may be particulate bed reactors having a bed of high permittivity particles between electrodes of electrically conducting material of the general type described in, for example, patent GB2274412. Alternatively, a dielectric barrier reactor (or so-called “silent discharge” reactor) or a reactor which has both dielectric barrier and a bed of high permittivity particles may be used. The requirement in accordance with the invention is to achieve an electric field strength within the plasma at least in excess of 10,000 volts/cm, preferably significantly higher, even up to and in excess of 80,000 volts/cm. This is achieved by attention to a combination of characteristics, namely the electrical potential applied, the spacing between the electrodes, the permittivity of the dielectric barrier and the permittivity of dielectric particles when used in the reactor.

FIG. 1 illustrates an adaptation of a dielectric barrier reactor in which a membrane for separating hydrogen produced in the reactor from other reaction products is incorporated within the reactor.

Referring to FIG. 1, the reactor 11 comprises a rectangular box enclosure formed between a ceramic cover plate 12 and a ceramic base plate 13. In this example, there are four electrode plates 14, 15, 16, and 17, each having an extension to provide an electrical contact, those, 18, 20, from electrode plates 14, 16 being on one side of the reactor 11, whilst the electrical contacts 19, 21 from the electrode plates 15, 17 are on the other side of the reactor 11.

Each electrode plate 14, 15, 16, 17 is secured with adhesive between respective pairs of ceramic plates 12, 22; 23, 24; 25, 26; and 27, 13. Where the electrode plates finish short of the edges of the ceramic plates, the space between respective ceramic plates is filled with ceramic adhesive, as for example referenced at 29. The ceramic plates serve as dielectric barrier layers for their associated electrode plates. Alumina is a suitable material for the ceramic dielectric barrier material. Aluminium nitride can also be used.

The dielectric barrier material can be a catalytic material, or contain a catalytic coating in or on its surface. The catalytic material can be produced by ion-exchange, doping, depositing by wet chemical techniques such as sol-gel processing, incipient wetness, by sputtering or by chemical vapour deposition or by thermal spraying for example by plasma spraying or by physical and chemical vapour deposition.

Ceramic spacers, one of which is referenced at 31, serve to hold the electrode plates spaced apart and also to form side walls of the rectangular box enclosure. At each end of the reactor 11, openings are provided at 32a, 32b and 32c for gas flow into and out of the reactor. At the outlet end of the reactor, separate openings are provided at 33a, 33b, 33c for product hydrogen gas flow out of the reactor.

The ceramic spacers (such as 31) are arranged also to support, in each of the three spaces between the four electrode plates, a layer of membrane 34a, 34b, and 34c, each of which, in this example, is a sheet comprising a ceramic such as zirconia or alumina. The membrane pore size is less than 2 nanometres. The membranes 34a, 34b, and 34c provide a surface which extends along the length of the respective gas flow path (32a, 32b and 32c).

These membranes may be in the form of different shapes in order to optimise the available surface for permeation of hydrogen therethrough. The membranes can have indentations with a square wave-form as shown in FIG. 1, but other shapes such as indentations with a triangular form can be used.

In operation, the reactor 11 is connected to the source of gaseous hydrocarbon and appropriate reforming reagents (steam, carbon dioxide or oxygen, according to the required reforming reaction) to undergo reforming. The gaseous reagents flow into the reactor at one end through the openings 32a, 32b, 32c and reaction products pass out of these openings at the other end of the reactor. Hydrogen formed in the reactor permeates through the membranes 34a, 34b, 34c and exits from the reactor 11 via the openings 33a, 33b, 33c.

An electrical power supply (not shown) is connected to apply between the pair of electrode plates 15, 17 and the pair of electrode plates 14, 16 a pulsed or alternating potential or direct current of the order of kilovolts to tens of kilovolts and (in the case of pulsed or alternating potential) repetition frequencies in the range 50 Hz to 50 kHz. The discharge gaps are narrow, that is of the order of 0.5 to 5 millimetres, the dimension transverse to the gap dimension being of the order of 10 to 200 millimetres. The small gap dimension is important in that a practical level of applied voltage can generate a plasma discharge in which the electric field strength has the required magnitude, as explained above. The plasma, together with any catalytic material incorporated in or on the dielectric barrier material, acts to promote the reforming reactions, the plasma providing sufficient energy input to avoid, or at least significantly reduce, the need to supply heat to the reactor.

Referring to FIG. 2, a second reactor for the plasma assisted reforming of hydrocarbon consists of a reactor chamber 300 which has inlet and outlet stubs 301, 302, respectively, by means of which gaseous reagents are introduced into the reactor and reaction products flow out.

Inside the reactor chamber 300 there is an inner electrode 303 which is supported within a dielectric tube 304, made for example out of α-alumina which has its upstream end closed by a spherical dome 305 to facilitate the flow of gaseous reagents through the reactor. The inner electrode 303 is supported in the dielectric tube 304 by two spider supports 306, 307. The inner surface of the dielectric tube can be metallised with a metal coating in order to increase the physical contact between the electrode and dielectric tube. The support 307 is connected to a high voltage input terminal 308 via a ceramic insulated feed 309 so that a potential of the order of kilovolts to tens of kilovolts and repetition frequencies in the range 50 to 50,000 Hz can be applied to the inner electrode 303. Concentric with the inner electrode 303 and dielectric tube 304 is a grounded outer electrode 310 made for example of stainless steel. The dielectric tube 304 and outer electrode 310 are supported within the reactor chamber 300 by disks 311, 312 made of an insulating ceramic material, such as alumina. A compliant heat resistant material 313 is interposed between the electrode support 311 and the dielectric tube 304.

As shown in FIG. 3, the outer electrode 310 has a series of baffles 314 and slots 315 315a. The baffles 314 extend from the outer electrode 310 to the inner surface of the wall of the reactor chamber 300 and act as grounding connections as well as causing the gaseous reagents to follow a convoluted path which has both axial, and circumferential components and being at least partially helical. There is also a radial component of flow, initially inwardly as the gaseous reagents transfer from outside the outer electrode 310 to the space between the electrodes 310 and 303 and then outwardly as the gas returns to exit from outside the outer electrode 310. Thus there is also a spiral component in the flow.

The baffle 314 is arranged to divide the space between the electrode 310 and the reactor chamber 300 into six segments. At the gas inlet end three of these segments are closed off at 314a, 314b and 314c to axial gas flow and the remaining three segments are open to axial gas flow into the space between the electrode 310 and the reactor chamber 300. These latter three segments are closed off by the baffle 314 at the gas outlet end of the reactor. Consequently the gas is forced to pass via slot 315 radially into the space between the electrodes 303 and 310 then passing in at least a partially helical manner before passing radially via the next slot 315a into the next segment of space between electrode 310 and reactor chamber 300. The baffle 314 leaves open this segment at the gas outlet end, allowing exhaust of the gaseous reaction products. Thus it will be seen that the gases both enter and leave the main part of the reactor 300 along the surface of the outer electrode 310 and the electrode supports 311, 312 have reliefs at their circumferences which are so positioned as to permit this to happen. Thus for a given gas velocity, the residence time of the gaseous reagents in the electric field is increased compared with either purely axial or radial flow. Note that in FIG. 3 part of the electrode 310 has been shown cut away at 316. This cut away is shown in the Figure only to illustrate the flow of the gases as they pass between the electrodes 303 and 310 and does not represent a structural feature of the reactor.

This form of reactor, as shown in FIGS. 2 and 3 can readily be provided with a bed of particulate high permittivity material filling the space between the electrode 303 (with its dielectric barrier covering tube 304) and electrode 310. The reactor in this form is then an illustration of a reactor which has both dielectric barrier and a bed of high permittivity particles.

It will be appreciated that the gaseous products emerging from the reactor of FIGS. 2 and 3 will require further treatment, as by membrane separation, to separate reaction products from one another, in particular to separate hydrogen from the other reaction products.

Use of a series spark gap can enhance the performance of the reactors. The electrical power supply (not shown) applies a discharge potential across the electrode plates 15, 17 and 14, 16 (FIG. 1) or between the inner electrode 303 and the grounded outer electrode 310 (FIG. 2). The operating voltage Vg of a dielectric barrier reactor of capacitance C2 is practically constant and represents the voltage required to break down the gas gap within the reactor with the formation of charge limited microdischarges. The value of Vg is related to the electric field at the surface of the dielectric barrier (or between dielectric particles or beads if the reactor is packed therewith) and determines the maximum energy that can be given to electrons in the microdischarges.

The value of Vg is pretty well determined, once the reactor geometry, packing, and gas operating temperature and pressure have been set.

It is possible to bring the reactor voltage V up to the value Vg faster if a series spark gap of capacitance Cl and breakdown voltage Vt is positioned in the supply line. If dV/dt is fast enough then the gap may overvolt before initiation, producing higher energy electrons in the microdischarge.

The Voltage across the reactor at any instant is:

V=Vs(C1/(C1+C2))

That is, the arrangement operates simply as a capacitative voltage divider.

If the spark gap capacitance is the same as the overall reactor capacitance i.e C1=C2, then V=Vs/2. Thus the supply voltage Vs would need to reach at least 2Vg before Vg could be attained across the reactor and microdischarges formed. If the spark gap is arranged to spark over at Vt, just lower than Vg and before discharge is initiated in the main reactor, then potentially up to 2Vg can be applied very quickly across the reactor itself. The application speed of this pulse can be expected to lead to beneficial overvolt conditions in the formation of the microdischarges, making them more effective.

The reactor will discharge in a few nanoseconds as the microdischarge runs its course and the voltage across C1 and C2 will fall switching off the spark gap. Using an AC power supply automatically repeats the cycle by recharging the ‘reactor+series spark gap’ to a new and slightly higher level as the AC voltage slowly increments. The voltage level has to be slightly higher for discharges subsequent to the first, because the capacitance C2 then has a base charge which has to be overcome.

It is envisaged that, instead of a spark gap and a reactor in series, one might use two dielectric barrier reactors in series, one with a large gap and the second with a smaller gap to fulfil the functions of C1 and C2 above.

The invention is not restricted to the details of the foregoing example(s). For instance additional energy, if required, can be supplied to the plasma by coupling microwave energy into the reactor chamber.

As mentioned above, enhanced formation of discharge streamers emanating from dielectric surfaces is achieved by providing on those surfaces exposed to gas an isolated discontinuous metallic film, preferably less than 10 μm in thickness and not connected to an external source of electric potential. The discontinuous metallic film may be in the form of spots or dots (of any circumferential shape, such as circular, rectangular or irregular, even fractal) of metal and can be present on one or more of the dielectric barriers. Such added metallic sources of discharge points can also be provided on or in high permittivity dielectric particles, where these are also provided in the gas flow path between the electrodes. Thus, for example such high dielectric packing material particles in the form of spheres or other shapes may be provided with surface deposits of thin film metal dots or spots. Alternatively, the packing itself may contain a distribution of metallic spheres (or other shapes) in amongst the dielectric particles.

Hydrogen produced in a process that uses a non-thermal plasma reactor can have many applications. For example in chemical synthesis, such as in Fischer-Tropsch reactions, in catalytic reactions both heterogeneous and liquid-phase catalysis, hydrogenation reactions and the synthesis of ammonia. The chemical synthesis may take place in a plasma reactor. It can also be used for the reduction of nitrogen oxides in the gaseous emissions from internal combustion engines. The hydrogen can be used as a gaseous source for fuel cells whose electrical output can be used for powering mobile vehicles on land, sea, air or in space such as cars, lorries, trains, ships aircraft and satellites as examples or for supplying electricity to stationary objects such as domestic appliances or for recharging batteries. Additionally, as a fuel for turbine generators and as a fuel for auxiliary back-up sources of power or as a fuel for direct injection into internal combustion engines. The hydrogen can be used in oil refinery applications for example to alter the octane levels of the products during refining of crude oils. Hydrogen and carbon monoxide in the product stream from a plasma reactor for the reforming of hydrocarbons can be used hydroformylation reactions for example the hydroformylation of alkenes that can be derived from refining operations. The hydrogen can also be contained in hydrogen-storage systems.

When reforming is carried out on long-chain hydrocarbons rather than short chain molecules such as methane, addition of hydrogen to the reactant entering the plasma can curtail the formation of polymeric hydrocarbon species and increase the yield of hydrogen product. It can be difficult to reform high molecular weight species, for example those found in diesel, by conventional reforming processes that do not involve the use of plasmas. A non-thermal plasma reactor can be used to crack diesel into smaller molecular species that can then undergo reforming conventionally in the absence of a non-thermal plasma reactor. Partial reforming can also take place resulting in the production of oxygenated hydrocarbons.

Other types of non-thermal plasma reactor can be used for the reforming of hydrocarbons. Corona reactors, an example of which is given in GB 2 282 738 B can be used. Microwave plasmas for example those generated by the use of electrodes that concentrate the microwave radiation into a plasma-forming region as described in GB 2 324 196 B can also be used for carrying out reforming reactions.

1. An apparatus for performing hydrocarbon reforming reactions, which apparatus comprises a reactor (11; 300) having an inlet duct (32a, 32b, 32c; 301) for feeding gaseous reagents to the reactor (11; 300) and an outlet duct (33a, 33b, 33c; 302) for reaction products, and, within the reactor (11; 300), electrodes (14, 15, 16, 17; 303, 310) and dielectric material (22-27; 304), a source of electrical potential connected in use of the apparatus to apply an electrical potential across the electrodes (14, 15, 16, 17; 303, 310), characterised in that the spacing and configuration of the electrodes (14, 15, 16, 17; 303, 310) and the dielectric material (22-27; 304) in relation to the applied electrical potential in use is such as to generate plasma in gaseous reagents passing through the reactor (11;300), in which plasma the electric field strength is in excess of 10,000 volts per centimetre.

2. An apparatus as claimed in claim 1, further characterised in that the electric field strength within the plasma is at least 20,000 volts per centimetre.

3. An apparatus as claimed in claim 1, further characterised in that the electric field strength within the plasma is at least 40,000 volts per centimetre.

4. An apparatus as claimed in claim 1, further characterised in that the electric field strength within the plasma is at least 80,000 volts per centimetre.

5. An apparatus as claimed in any of the preceding claims, wherein the reactor (11; 300) is a dielectric barrier reactor having at least one electrode (14, 15, 16, 17; 303) with a dielectric barrier layer (22-27; 304) in intimate contact with its surface or surfaces which face the other electrode, further characterised in that the spacing between the electrodes (14, 15, 16, 17; 303, 310) is sufficiently small for the applied potential across them to generate plasma having the said electric field strength.

6. An apparatus as claimed in any of the preceding claims, further characterised in that the reactor (11) is provided with a plurality of electrodes (14, 15, 16, 17) arranged in pairs and electrical potential is applied across each pair of electrodes.

7. An apparatus as claimed in claim 6, further characterised in that all the electrodes (14, 15, 16, 17) are coated with a dielectric barrier layer.

8. An apparatus as claimed in any of claims 1 to 6, further characterised in that a bed of high permittivity dielectric particles fills the space between electrically conducting surfaces of the electrodes (14, 15, 16, 17; 303, 310), the permittivity of the particles serving to concentrate the electric potential at the contact points between the particles such that the aforesaid electric field strength is achieved in plasma discharges formed in the interstices between the particles.

9. An apparatus as claimed in any of claims 1 to 7, wherein the dielectric barrier layer comprises alumina.

10. An apparatus as claimed in claim 8, wherein the particles comprise particles of barium titanate or calcium titanate or high permittivity dielectric material.

11. An apparatus as claimed in claim 8 or claim 10, wherein the particles are in the form of spheres, pellets, extrudates, fibres, sheets, wafers, frits, meshes, coils, foams, honeycomb monoliths or granules or as a coating on any of the above shapes or contained within a dielectric, polymeric or metallic material in any of the above shapes or as a combination of more than one of the aforementioned forms of particulate material.

12. An apparatus as claimed in any of the preceding claims, further characterised in that there is included a membrane positioned to receive output gaseous product from the reactor (11, 300) to separate hydrogen gas therefrom.

13. An apparatus as claimed in claim 12, further characterised in that the membrane is outside the reactor (11, 300).

14. An apparatus as claimed in claim 12, further characterised in that the membrane (34a, 34b, 34c) is positioned within the reactor (11) and is exposed to the plasma.

15. An apparatus as claimed in claim 14, further characterised in that the membrane (34a, 34b, 34c) is provided by one or more of the electrodes in the reactor chamber, which electrode or electrodes is or are in the form of a gas permeable electrically conducting material which itself is permeable to hydrogen but not other reaction products.

16. An apparatus as claimed in claim 14, further characterised in that the membrane (34a, 34b, 34c) is provided by one or more of the electrodes in the form of a gas permeable electrically conducting material having a coating thereon of material which is permeable to hydrogen but not other reaction products.

17. An apparatus as claimed in any of the preceding claims, wherein catalytic materials are incorporated in the apparatus to assist the desired reforming reactions.

18. A method of reforming hydrocarbon comprising forming within a gaseous reaction mixture including the hydrocarbon a plasma in which the electric field strength exceeds 10,000 volts per centimetre.

19. A method of reforming hydrocarbon comprising forming within a gaseous reaction mixture including the hydrocarbon a plasma in which the, electric field strength is at least 20,000 volts per centimetre.

20. A method of reforming hydrocarbon comprising forming within a gaseous reaction mixture including the hydrocarbon a plasma in which the electric field strength is at least 40,000 volts per centimetre.

21. A method of reforming hydrocarbon comprising forming within a gaseous reaction mixture including the hydrocarbon a plasma in which the electric field strength is at least 80,000 volts per centimetre.
